(19) United States
(12) Patent Application Publication
(10) Pub. No.: US 2026/0083416 A1
(43) Pub. Date: Mar. 26, 2026

(54) Graphical User Interface for Flow Rate Extraction from Angiographic Information

(71) Applicant: AngioInsight, Inc., Ann Arbor, MI (US)

(72) Inventors: Srinivas Paruchuri, Minneapolis, MN (US); Yang Zhou, St. Louis Park, MN (US); Scott Burger, Maple Plain, MN (US); Chris Ernst, Colorado Springs, CO (US); Judith Gonzalez, Raleigh, NC (US); Abdulhak Qamili, Skopje (MK); Todd Villines, Charlottesville, VA (US)

(73) Assignee: AngioInsight, Inc., Ann Arbor, MI (US)

(21) Appl. No.: 18/892,184

(22) Filed: Sep. 20, 2024

(51) Int. Cl. A61B 6/46; A61B 6/50; G06T 11/20

(52) U.S. Cl. CPC A61B 6/465; A61B 6/467; A61B 6/507; G06T 11/26; G06T 2200/24; G06T 2210/41

(57) ABSTRACT

A computer-implemented method and system for assessing vascular disease is disclosed. The disclosure provides receiving angiography image data, including a plurality of image frames captured over a sampling time-period for a subject; identifying a representative image frame from the plurality of image frames; segmenting the plurality of image frames to isolate a vessel region; inferring a plurality of centerline node points associated with a centerline of the vessel; tracking movement of the plurality of centerline node points between successive centerline node points of the plurality of angiogram image frames; registering each segmented frame of the plurality of image frames to the representative image frame; and determining a flow rate of the vessel based in part on a change in length of the vessel represented in successive registered image frames. In embodiments, resultant measurements and results may be displayed upon one or more user interfaces.

### TECHNICAL FIELD

The present disclosure pertains to computer-assisted methods, systems and infrastructure for providing a quantitative analysis of blood flow through vasculature of a patient or subject. Embodiments described herein generally relate to determining a rate of blood flow in a vessel from a series of angiographic images of the vessel.

### BACKGROUND

Coronary Artery Disease (CAD) is characterized by plaque build-up from atherosclerosis in the coronary arteries. CAD affects the main blood vessels that supply blood to the heart and often results in stenosis (or narrowing) and/or blockage of coronary arteries, which can lead to symptoms such as angina, myocardial infarction, etc.

CAD may also be referred to as coronary heart disease. Symptoms of CAD may not acutely manifest early in the disease progression. For example, symptoms of coronary artery disease may not immediately be perceived by those afflicted but may instead only appear during intervals of intense exertion, such as, during exercise. However, as the disease progresses and the coronary arteries continue to narrow through atherosclerosis, symptoms of CAD may become more frequent.

Additionally, many symptoms of CAD may not present until the coronary arteries are sufficiently narrowed and/or obstructed. Early detection of CAD onset may provide opportunities for alternative and/or less invasive interventions than when CAD is not detected early. As such, early detection may extend a patient's health span and/or improve their quality of life.

CAD is often diagnosed with a combination of imaging modalities. For example, Angiography is a foundational medical imaging technique for visualizing blood vessels and organs and is often used to identify abnormalities related to CAD. Angiography involves imaging (e.g., under X-ray-based techniques such as fluoroscopy or the like) the arteries and veins while a contrast agent visible to the imaging modality (e.g., radio-opaque) is injected into the patient's arteries (sometimes referred to as an arteriogram) and veins (e.g., venogram) via a catheter. Angiography is instrumental in diagnosing and managing various vascular conditions. While often used as a generic term for use in arteries and veins alike, angiography performed on an artery is sometimes referred to as arteriography and when performed on a vein may be referred to as venography.

However, as noted above, symptoms of CAD often do not present early. As such, angiography is often applied after a patient seeks treatment due to late-occurring symptoms of CAD. Further, other more invasive imaging and/or measurement modalities may be used to confirm the CAD diagnosis (e.g., intravascular imaging, pressure measurement, etc.) Further, as some methods for diagnosing CAD are invasive, they are often not carried out until sufficient symptoms of the disease present.

There exist more quantitative approaches to assessing the severity of a stenosis, such as for example, the use of Fractional Flow Reserve (FFR) techniques. FFR is a metric defined as the ratio between hyperemic flow in an artery with a stenosis and the expected hyperemic flow in the same artery without the stenosis. In a coronary artery, for example, FFR can be expressed as the ratio of a coronary pressure distal to a stenosis to a coronary pressure proximal to that stenosis. Determining the FFR for an artery conventionally requires invasive catheter-based pressure measurements. Although non-invasive methods and approaches for determining FFR have been developed, physicians often choose not to perform non-invasive FFR analyses due to the additional cost to the procedure and/or limitations of the accuracy of the analyses.

Unfortunately, this exacerbates the problem identified above in that CAD is often not diagnosed early. In fact, conventional methods and systems are unable to provide early beneficial detection of CAD using non-invasive techniques. Therefore, an unmet need exists in the art for reliable, accurate and rapid assessment of CAD and related vascular disease with non-invasive techniques. That is, a need exists in the art for real-time determination of vascular disease from angiographic imaging data and related angiographic data that allows a physician (or other user) to quickly assess a state of vascular disease within a patient, and with the capability to do so before late-onset symptoms of vascular disease manifest. A further concern regarding the diagnosis of CAD, is that conditions and/or anomalies may develop in the smaller vessels that branch from the coronary arteries, such as within the microvasculature and surrounding vessels. If present anomalies are not detected at this stage, microvascular disease (MVD) may develop, which is a known precursor to CAD.

As such, angiography is also utilized in diagnosing and managing of various other vascular conditions, including allowing for intricate assessment of MVD, a condition affecting the smallest blood vessels of the body. Although there exist systems, methods and approaches to analyzing angiographic images for the purposes of assessing conditions of the vasculature, it is to be appreciated that these current systems, methods and approaches are limited. In many respects, and partially due to the constraints of the procedural environment, obtaining real-time measurements and assessments of vascular conditions from angiographic images remains a difficult time-consuming and data-consuming process that does not always ensure accuracy and effectiveness of measurement and assessment. Thereby demonstrating an unmet need in the art as the morbidity of vascular disease is heavily dependent on the timing of detection and diagnosis; whereby early detection of a vascular condition or anomaly within a patient's microvasculature can improve the prognosis, outcome and treatment plan for a patient susceptible to vascular disease and related conditions.

Overall, microvascular disease (MVD) can lead to significant morbidity if not accurately diagnosed and treated and is often difficult to detect using conventional techniques such as angiography alone. MVD is alternatively referred to as coronary MVD, microvascular endothelial dysfunction, small artery disease, or small vessel disease. In general, MVD is a disease that affects the walls and inner lining of smaller blood vessels, such as arterioles, which branch off from larger blood vessels in the vasculature.

For example, in MVD, the coronary artery blood vessels that branch off from the larger coronary arteries may present damage or narrowing along the inner walls. Such narrowing (e.g., due to plaque formation or the like) can inhibit or block blood flow to the heart. Damage to the inner walls of these vessels can further lead to spasms which may also decrease blood flowing to the heart. Additionally, several abnormalities (e.g., convexities, plaque formations, strictures, and the like) in these smaller arteries may contribute to MVD.

MVD often presents significant challenges in diagnosis due to the subtlety of its initial manifestations. Traditionally, signs of MVD are not overtly visible on standard angiograms. They might only become apparent under stress tests, which can indicate reduced efficiency in blood flow during increased demand put upon the vasculature. However, these indications are usually detected only after the disease has progressed to a more advanced stage, making early intervention more difficult.

Women more frequently develop MVD than men and it occurs particularly in younger women. The risk factors for MVD are the same as for coronary artery disease, including diabetes, high blood pressure and high cholesterol. Diagnosing MVD and other forms of vascular disease can be extremely challenging.

Positron Emission Tomography (PET) scans and other types of imaging (i.e., angiographic imaging) may be employed to measure blood flow through the larger blood vessels. However, these methods still fall short in accurately measuring blood flow through the smaller branching blood vessels. As such, these imaging modalities are not sufficient to objectively and quantitatively assess the risk, presence and/or prevalence of MVD.

Traditional angiography primarily offers a qualitative analysis of blood flow, pinpointing the location of blockages or strictures but fails to provide a quantitative assessment on blood flow velocity data. Such data are vital for assessing the severity of vascular impairments, including those in the microvasculature, which are often more challenging to detect and quantify due to their small size and complex fluid mechanics. The lack of quantitative flow velocity data can hinder prompt and accurate diagnosis and treatment of microvascular disease, necessitating additional tests that may delay essential therapeutic interventions.

Thus, there exists an unmet need for less invasive techniques to accurately assess CAD and MVD. Ideally, these techniques should be implementable in real-time and offer optimal and early beneficial detection of these diseases.

### SUMMARY

As stated above, angiography provides an analysis of blood flow and can be used to pinpoint the location of stenosis or blockages in arteries and veins. However, assessment of angiographic videos, images and related data is somewhat subjective and fails to provide reliable quantitative data on parameters such as, e.g., blood flow velocity in the vasculature. In assessing CAD, MVD, or other vasculature conditions, physicians often desire to review quantitative measures of vascular blood flow to assess the severity of the disease and determine treatment options. However, quantitative assessments, such as, for example FFR, either are not performed due to their invasive nature and/or cannot be performed in all the microvascular structure in which a physician may want to assess for disease.

The present disclosure provides methods and systems that substantially advance the diagnostic capabilities of angiogram videos, images and related, additional and/or complementary angiographic or health information of a subject (e.g., in some instance referred collectively or individually as “angiographic data”), and provides qualitative and quantitative measures of blood flow parameters and related information therefrom. For example, the disclosure provides a real-time determination of blood flow velocity from a series of angiographic images. With the present disclosure, physicians may be able to assess the status, stage, condition, prevalence, percentage, diagnosis, prognosis, and/or manifestation of CAD, MVD, and other like and/or related conditions using the disclosed measures derived from angiography images.

In particular, the present disclosure provides methods and systems to determine fluid flow rate through anatomical structures including arteries and veins. Details of the methods and systems described herein are described in greater detail below. However, in general, the present disclosure provides methods and systems to determine flow rates based on the movement of contrast dye through microvascular structure across a series of angiographic images.

Accordingly, the present disclosure provides quantitative datapoints directly from angiogram images that can be used by a physician to augment the more subjective assessments made from the angiographic data. This can provide an advantage over conventional imaging and image analysis techniques by offering immediate, actionable data for healthcare providers. As another example, the present disclosure provides methods and system to quantify microvascular flow dynamics and improve the diagnosis and management of microvascular disease by facilitating a more accurate assessment of these critical, and often overlooked, components of the vascular system.

The present disclosure can be implemented to provide methods and systems to enhance the precision of diagnoses, such as diagnoses of various states of CAD, vascular disease, and/or microvascular disease, which can enable more tailored treatments and potentially reduce the need for subsequent testing.

With some embodiments, the present disclosure can be implemented as part of and/or integrated into an angiogram acquisition system and can provide flow velocity measurements directly from the angiogram image data.

In one example, the disclosure provides methods and systems for computer-assisted quantitative analysis of blood flow through vasculature of a patient or subject. Some embodiments of the disclosure can be implemented as a computer implemented method or as part of an angiographic data acquisition or analysis system. In such examples, the computer implemented method can identify vasculature of a patient represented in frames of an angiographic video (i.e., a blood vessel, a blood vessel branch, a system or series of blood vessels, a system or series of blood vessel branches, or the like). The vasculature can be tracked across the frames and a volume of the vessel can be identified at different points in a sampling period. The volume of the vessel over the sampling period can be correlated to a rate of blood flow in the vasculature. This example, as well as various details of the example, are described in greater detail below.

In a first example, a computer-implemented method for assessing blood flow in a vessel and/or for assessing vascular disease is provided. A computer-implemented method for assessing blood flow in a vessel and/or for assessing vascular disease may include: receiving, by a processor, angiography image data for a vessel of a subject. The angiography image data may include a plurality of image frames captured over a sampling time-period. The plurality of image frames may show that contrast dye is present and may be captured over a sampling time-period. The computer-implemented method of this and other examples may further include identifying, by the processor via one or more computer vision algorithms, a representative image frame from the plurality of image frames. The computer-implemented method of this and other examples may further include segmenting, by the processor, the plurality of image frames to isolate the vessel based in part on mapping the plurality of image frames onto the representative image frame to generate a plurality of segmented image frames. The computer-implemented method of this and other examples may further include inferring, by the processor for the representative image frame via a centerline identification machine learning (ML) model, a plurality of centerline node points associated with a centerline of the vessel. The computer-implemented method of this and other examples may further include tracking, by the processor, movement of the plurality of centerline node points between adjacent centerline node points of the plurality of angiogram image frames. The computer-implemented method of this and other examples may further include aligning or stabilizing, by the processor, each frame of the plurality of segmented image frames to the frame of the segmented image frames associated with the representative image frame based on the movement of the plurality of centerline node points between successive centerline node points of the plurality of segmented image frames. The computer-implemented method of this and other examples may further include determining, by the processor, a flow rate of the vessel based in part on a change in length of the vessel represented in successive image frames of the stabilized image frames. The vessel of this and other examples may contain one or more dyes such as contrast media or the like.

Alternatively, or additionally, the computer-implemented method of this and other examples may further include plotting growth of the centerline of the vessel over the sampling time-period based on the plurality of node points.

Alternatively, or additionally, the computer-implemented method of this and other examples may further include determining a change in length of the vessel between adjacent frames of the plurality of image frames based on the plot of the growth of the centerline. The computer-implemented method of this and other examples may further include approximating the cross-sectional area of the change in length of the vessel based on a calculated radius of the vessel, and deriving a change in volume of the vessel based in part on the cross-sectional area.

Alternatively, or additionally, the computer-implemented method of this and other examples may further include applying pre-processing, by the processor, to the plurality of image frames. The pre-processing may include de-noising, linear filtering, image size normalization, and/or pixel intensity normalization.

Alternatively, or additionally, the computer-implemented method of this and other examples may further include feeding the pre-processed plurality of image frames to an angiography processing network (APN) and a backbone segmentation network. The APN may be trained to remove artifacts from the angiographic image data.

Alternatively, or additionally, the computer-implemented method of this and other examples may further include iteratively mapping the centerline of the vessel, starting with the representative segmentation, from a frame (i−1) to a frame (i) based on movement of the centerline node points between the frame (i−1) and the frame (i) to align each segmentation of the plurality of image frames to the representative segmentation.

Alternatively, or additionally, the computer-implemented method of this and other examples may further include inferring, via one or more ML models, one or more positional data points from the plurality of image frames; and generating, via the one or more ML models, a plurality of two-dimensional (2D) segmented vessel images based upon the inferred one or more positional data points.

Alternatively, or additionally, the computer-implemented method of this and other examples may further include mapping, via the one or more ML models, the 2D segmented vessel images into a three-dimensional coordinate system based upon the inferred one or more positional data points and generating a three-dimensional (3D) model of the vessel.

Alternatively, or additionally, the computer-implemented method of this and other examples may further include inferring, via one or more ML models based upon the determined flow rate in the vessel, at least one of the presence or absence of a vascular occlusion, size of a vascular occlusion, and morphology of a vascular occlusion.

Alternatively, or additionally, the computer implemented method of this and other examples may further include truncating one or more centerline node points from one or more centerlines associated with each segmented image frame based on a computer vision algorithm to form a set of truncated centerlines. The computer implemented method of this and other examples may include determining the area of the vessel represented in successive ones of the aligned image frames based on the set of truncated centerlines.

Alternatively, or additionally, the computer-implemented method of this and other examples may further include determining a change in length of one or more vessel segments of the vessel from the set of truncated centerlines, approximating a cross-sectional area of the one or more vessel segments based on a derived radius of the vessel, and determining the area of the vessel based on the change in length and the cross-sectional area.

Alternatively, or additionally, the computer-implemented method of this and other examples may further include applying, by the one or more processors, to the received angiographic image data at least one of a de-noising process, a linear filtering process, an image size normalization process, and a pixel intensity normalization process.

Alternatively, or additionally, the computer-implemented method of this and other examples may be implemented in a computing system for assessing blood flow in a vessel and/or for assessing vascular disease. In this and other examples, the computing system may include a processor and a memory storage device. The memory storage device may include instructions, that when executed by the processor, cause the processor to implement any of the computer-implemented methods disclosed herein.

Alternatively, or additionally, the computer-implemented method of this and other examples may further include determining the change in length of one or more vessel segments of the vessel from the plot of centerline node points and approximating the cross-sectional area based on a calculated radius of the vessel; and calculating the volume change over time through the vessel.

Alternatively, or additionally, the computer-implemented method of this and other examples may further include stabilizing each segmentation frame of the plurality of image frames received from the image data by matching tracking points between image frames via one or more machine-learning (ML) models.

Alternatively, or additionally, the computer-implemented method of this and other examples may further include feeding the pre-processed angiographic image data to an angiography processing network (APN) and a backbone semantic segmentation network. The APN may be trained to remove artifacts from the angiographic image data.

Alternatively, or additionally, the computer-implemented method of this and other examples may further include feeding the pre-processed angiographic image data to an angiography processing network (APN) and a backbone segmentation network. In this and other examples, the APN may be trained to remove artifacts from the angiographic image data.

Alternatively, or additionally, the computer-implemented method of this and other examples may be executed by a system for assessing blood flow in a vessel and/or vascular disease. The system may include a processor and a memory storage device. The memory storage device may include instructions that when executed by the processor, cause the processor to implement any one of the computer-implemented methods described herein.

### BRIEF DESCRIPTION OF THE DRAWINGS

The disclosure may be more completely understood in consideration of the following detailed description in connection with the accompanying drawings, in which:

FIG. 1 illustrates a system to derive a blood flow rate in a vessel from angiographic images, in accordance with at least one embodiment.

FIG. 2 illustrates a logic flow to derive a blood flow rate in a vessel from angiographic images, in accordance with at least one embodiment.

FIG. 3 illustrates a machine learning (ML) architecture, in accordance with at least one embodiment.

FIG. 4 illustrates a logic flow to generate 2D segmented vessel images from angiography image data, in accordance with at least one embodiment.

FIG. 5 and FIG. 6 illustrate example convolutional neural network (CNN) frameworks, in accordance with at least one embodiment.

FIGS. 7A, 7B, 7C, 7D, 7E, and 7F illustrate angiographic image frames of the heart and associated vessels.

FIGS. 8A, 8B, 8C, 8D, 8E, and 8F illustrate segmented 2D image frames corresponding to the angiographic image frames of FIGS. 7A, 7B, 7C, 7D, 7E, and 7F, in accordance with at least one embodiment.

FIG. 9 illustrates the tracked points of a vessel structure and associated tracking points represented in the angiographic image frames, in accordance with at least one embodiment.

FIGS. 10A, 10B, 10C, 10D, 10E, and 10F illustrate outputted tracking points overlaid onto image frames associated with various stages of contrast dye saturation within a vessel structure, in accordance with at least one embodiment.

FIGS. 11A, 11B, 11C, 11D, 11E, and 11F illustrate an example process of angiographic image frame segmentation of FIGS. 10A, 10B, 10C, 10D, 10E, and 10F, in accordance with at least one embodiment.

FIG. 12 illustrates a plot of the normalized flow rate over time, in accordance with at least one embodiment.

FIG. 13 illustrates a plot of normalized pixel counts over time, in accordance with at least one embodiment.

FIGS. 14A, 14B, 14C, 14D, and 14E illustrate an example graphical user interface (GUI) and graphical elements of the GUI in accordance with at least one embodiment.

FIGS. 15A and 15B illustrates example graphical elements in accordance with embodiments.

FIG. 16 illustrates an example GUI and graphical elements of the GUI.

### DETAILED DESCRIPTION

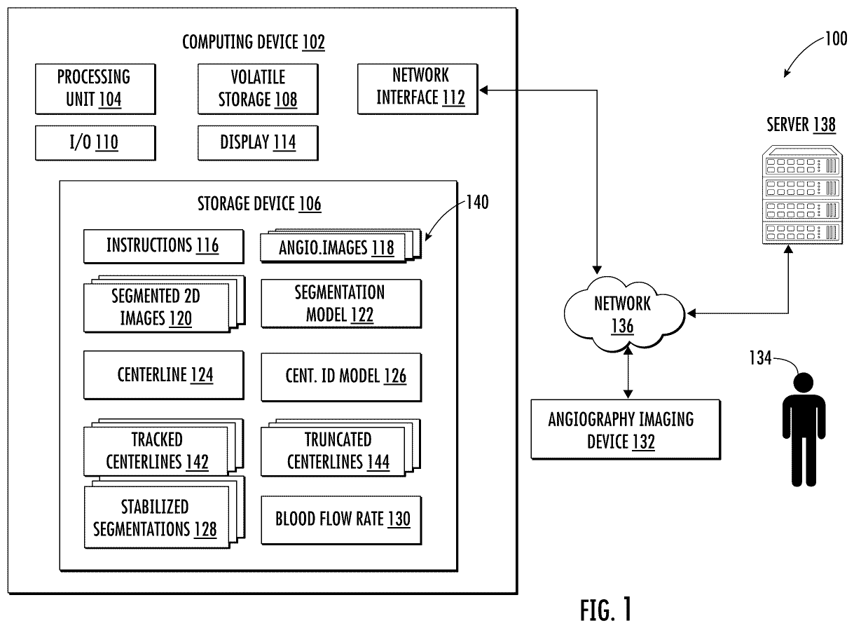

FIG. 1 illustrates a blood flow assessment system 100 that can be provided according to embodiments of the present disclosure. Blood flow assessment system 100 includes a computing device 102 configured to determine blood flow rate 130 for vasculature structure represented in angiographic images 118. Computing device 102 can be any of a variety of computing devices. In some embodiments, computing device 102 can be incorporated into and/or implemented by a console of an angiographic acquisition device. With some embodiments, computing device 102 can be a workstation or server communicatively coupled to an angiographic imaging device 132 directly, indirectly, or through a network (i.e., wireless network, wired network, Local Area Network (LAN) or the like). With still other embodiments, computing device 102 can be provided by a cloud-based computing device, such as, by a computing as a service system (CaaS) accessibly over a network (e.g., the Internet, an intranet, a wide area network, or the like). Alternatively, or additionally, computing device 102 can be provided by a software-based computing device, such as, by a software as a service system (SaaS).

Computing device 102 can include processing unit 104, storage device 106, volatile storage 108, input and/or output devices 110, network interface 112, and display 114. Processing unit 104 may include circuitry or processor logic, such as, for example, any of a variety of commercial processors. In some examples, processing unit 104 may include multiple processors, a multi-threaded processor(s), a multi-core processor(s) (whether the multiple cores coexist on the same or separate dies). Additionally, in some examples, the processing unit 104 may include separate processing circuitry (e.g., graphics processing units, neural processing units, or the like) and may include dedicated memory and/or processing pipelines. In some examples, the processing unit 104 can be a central processing unit (CPU) and/or a graphics processing unit (GPU). In other examples, the processing unit 104 can be an application specific integrated circuit (ASIC) or a field programmable gate array (FPGA).

The storage device 106 may include logic, a portion of which includes arrays of integrated circuits, forming non-volatile memory to persistently store data while volatile storage 108 includes circuitry configured to non-persistently store data (e.g., volatile memory). It is to be appreciated that storage device 106 and volatile storage 108 may be based on any of a variety of technologies and can for example, be formed from one or more types of memory, such as, for example, dynamic random access memory (DRAM), NAND memory, NOR memory, or the like.

Input and/or output devices 110 can be any of a variety of devices to receive input and/or provide output. For example, input and/or output devices 110 can include a keyboard, a mouse, a joystick, a peripheral device such as a printer or the like, a foot pedal, a microphone/speaker, a display, a touch enabled display, a brain-computer interface (BCI), a haptic feedback device, an LED, an OLED or the like, and combinations of any of the foregoing. Alternatively, or additionally, output modalities may include the generation of reports, data and/or files in various formats, including but not limited to PDF, DOCX, JPEG, GIF, or other like and/or suitable formats known in the art.

Network interface 112 can include logic and/or features to support a communication interface. For example, network interface 112 may include one or more interfaces that operate according to various communication protocols or standards to communicate over direct or network communication links, such as network 136. Direct communications may occur via use of communication protocols or standards described in one or more industry standards (including progenies and variants). For example, network interface 112 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 112 can include logic and/or features to enable communication over a variety of wired or wireless network standards (e.g., 802.11 communication standards). For example, network interface 112 may be arranged to support wired communication protocols or standards, such as Ethernet or the like. As another example, network interface 112 may be arranged to support wireless communication protocols or standards, such as, for example, Wi-Fi, Bluetooth, ZigBee, LTE, 5G or the like. Computing device 102 will include various busses, communication, and power distribution components known in the art, but which are not shown for brevity and clarity of presentation. Display 114 can include any of a variety of computer displays, including but not limited to touch displays, haptic feedback displays, monitors, smartphones, tablets, brain-computer interfaces (BCI), manual-input displays, or the like.

Storage device 106 may include any number of components in various combinations, including, for example, instructions 116, angiographic images 118, segmented 2D images 120, segmentation mode 122, centerlines 124, a centerline ID model 126 (e.g., a computer skeletonization model, or the like), stabilized segmentations 128, and blood flow rate 130. Angiographic images 118 may include but are not limited to angiographic images formatted according to a medical imaging standard, such as, for example, the Digital Imaging and Communications in Medicine (DICOM) standard promulgated by the Medical Imaging Technology Association (MITA), a division of the National Electrical Manufacturers Association (NEMA).

In general, processing unit 104 can execute instructions 116 to determine blood flow rate 130 based on movement of the contrast dye across the frames of the angiographic images 118. For example, processing unit 104 can execute instructions 116 to identify (e.g., via one or more frame selection algorithms and/or computer vision algorithms) a representative frame 140 from the frames of the angiographic images 118. It is noted that the representative frame 140 can be any one of angiographic images 118 (e.g., 118(1), 118(2), 118(n), etc.)

Processing unit 104 can execute instructions 116 to generate segmented 2D images 120 from angiographic images 118 to isolate a vessel region represented in each frame. A vessel region may constitute a blood vessel branch, a blood vessel section, a blood vessel bifurcation, a series of blood vessels, an artery, arteries, an arteriole, arterioles, a capillary, capillaries, a venule, venules, the like, or any combination of the aforementioned. With some embodiments, processing unit 104 can execute instructions 116 to infer segmented 2D images 120 from angiographic images 118 and the representative frame using a machine learning (ML) model, such as, for example segmentation model 122.

Processing unit 104 can execute instructions 116 to identify a centerline for the representative frame 140 of the angiographic images 118 based in part on the corresponding frame from the segmented 2D images 120. The centerline 124 will include a node point or node points defining the centerline. For example, in some embodiments processing unit 104 can execute instructions 116 to identify the centerline 124 for the representative frames 140 from the corresponding segmented 2D image 120. As another example, processing unit 104 can execute instructions 116 to identify centerlines 124 by applying image processing techniques to generate a skeletonization of the corresponding segmented 2D image 120 and deriving the centerline 124 from the skeletonization. In other examples, processing unit 104 can execute instructions 116 to infer the centerline 124 for the representative frame 140 from a centerline identification model 126 and segmented 2D image 120.

Processing unit 104 can execute instructions 116 to align each frame from the segmented 2D images 120 to the representative frame 140 of the angiographic images 118 based on movement of the node points from frame-to-frame across the frames of angiographic images 118. For example, processing unit 104 can execute instructions 116 to “track” the movement of the node points of each centerline from frame-to-frame. With some examples, processing unit 104 can execute instructions 116 to apply a point tracking algorithm (e.g., persistent independent particles (PIPs++) tracking algorithm, or the like) to track movement of a feature (or features) of the image associated with each respective node point across the frames in angiographic images 118. For example, processing unit 104 can execute instructions 116 to track the centerline 124 across the angiographic images 118 (e.g., using the PIPs++ algorithm, or the like) to generate tracked centerlines for all frames (or rather all frames but the representative frame 140). This is described in greater detail below.

Processing unit 104 can execute instructions 116 to align each frame from the segmented 2D images 120 to the frame of the segmented 2D images 120 associated with the representative image frame 140 based on the tracked movement of the node points of the centerlines 124 to form a set of stabilized images 128. For example, the segmented 2D images can be aligned, beginning with the representative frame, and continuing to each prior (or subsequent in time) frame based on the truncated centerlines 144. As such, processing unit 104 can execute instructions 116 to remove movement of the vessel in the x-y plane (i.e., lateral movement, radial movement, etc.) across the frames in the segmented 2D images 120. As such, movement due to for example, patient breathing, heart beats, anatomic oscillations, etc. is counteracted in the stabilized images 128. Further, processing unit 104 can execute instructions 116 to overlay the tracked centerlines 142 onto corresponding segmented 2D images 120 and truncate centerlines based on the segmentations to generate truncated centerlines 144.

Processing unit 104 can execute instructions 116 to determine a flow rate of the vessel represented in the angiographic images 118 based at least in part on a change in length of the segmented vessel from frame-to-frame and change in percentage of visible vessel from frame-to-frame across the stabilized images 128 as may be dictated by the level of saturation of contrast dye within the vessel. In general, processing unit 104 can execute instructions 116 to derive the growth of the centerline (e.g., as represented by the contrast dye in the segmented 2D images 120) as a function of time (e.g., based on the frame rate) to determine the flow rate in the vessel. For example, processing unit 104 can execute instructions 116 to derive the volume of the vessel at each frame, based on the truncated centerlines 144, and further to derive a change in volume from frame-to-frame and derive the flow rate as the change over time (e.g., over the frame rate).

As described further herein, flow rate of fluid (e.g., blood) through the vessel may be calculated by measuring the change in length of the vessel segments and approximating the cross-sectional area as a circle based on a nominal and/or local and/or constant and/or pixel-width and/or pixel-density radius. Alternatively, or additionally, flow rate of fluid (e.g., blood) through the vessel may be calculated by measuring the change in length of the vessel segments and approximating the cross-sectional area as a circle based on a radius defined and/or determined by pixel density, pixel size, pixel width, and/or any dimension and/or quantitative amount of pixels. Processing unit 104 can execute instructions 116 to calculate the volume change over time by integrating the areas of the cross-sections along the length of the centerline, for instance and by non-limiting examples, by applying the principles of cylindrical shell integration and/or Riemann summation. Plotting this change allows for the determination of flow rate over time, with the peak rate serving as a proxy for overall flow rate. This method, although based on 2D projections, can be refined through analysis from multiple angles to select the highest flow rate as the representative flow rate.

In some embodiments, blood flow assessment system 100 can be integrated into a catheterization laboratory (e.g., cath-lab, or the like) or other imaging clinic and a flow rate derived in real time as angiographic images are captured. As such, in some examples, processing unit 104 can execute instructions 116 to cause computing device 102 to receive angiographic images 118 from angiography imaging device 132. Angiography imaging device 132 itself can include an imaging table on which a patient 134 can be positioned and an imaging device (e.g., c-arm imager, or the like) configured to capture images of the arteries and/or veins (of the patient) while contrast is injected into the vasculature of the patient 134. Processing unit 104 can execute instructions 116 to receive information elements (e.g., data structures, or the like) from angiography imaging device 132 (e.g., via network 136, or the like) comprising indications of angiographic images 118 (e.g., using the DICOM standard, or the like).

In other embodiments, blood flow assessment system 100 can be configured to access (e.g., from a local, remote, or cloud-based memory storage device) the angiographic images 118 after capturing the angiographic image data. For example, blood flow assessment system 100 can include a server 138 configured to store angiographic images 118 and processing unit 104 can execute instructions 116 to receive angiographic images 118 from server 138 (e.g., via network 136, or the like). As noted above, angiographic images 118 comprise several image frames captured over a time-period.

With some embodiments, processing unit 104 can execute instructions 116 to generate a graphical element comprising an indication of the normalized flow rate over time for display on display 114. Suitable graphical elements may include, but are not limited to windows (i.e., an area on the screen that displays information), container windows, browser windows, text terminals, child windows, message windows, menus, user menus, menu bars, context menus, menu extras, controls, widgets, icons, tabs, cursors, text cursors, pointer cursors, selection tools, handles, adjustment handles or the like. In other examples processing unit 104 can execute instructions 116 to generate a report comprising indications of the normalized flow rate over time. The report can be provided to a user (e.g., physician, clinic, medical records custodian, insurance provider, etc.) via network 136, or the like.

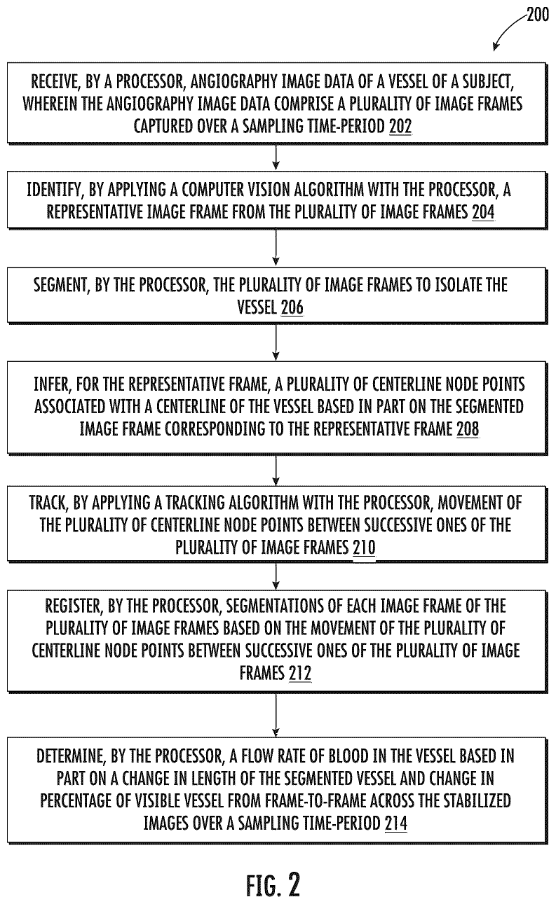

FIG. 2 illustrates a logic flow 200 that can be implemented according to the present disclosure. With some examples, blood flow assessment system 100 can be configured to implement logic flow 200. Although logic flow 200 is described with reference to blood flow assessment system 100, examples are not limited in this context. Logic flow 200 can begin at block 202. At block 202 (“receive, by a processor, angiography image data of a vessel of a subject, wherein the angiography image data comprise a plurality of image frames captured over a sampling time-period”), a time series of frames of angiographic image data can be received, typically but not necessarily moving forward in increments of time. In general, the angiographic image data will comprise indications, or be representative of a coronary vessel or coronary vasculature or microvasculature of the patient. For example, processing unit 104 can execute instructions 116 to receive angiographic images 118 (e.g., from angiography imaging device 132, from server 138, or the like) where angiographic images 118 represents a coronary vessel or coronary vasculature of the patient 134. As used here, the term vessel is representative of arteries, veins, coronary vessels and coronary vasculature. Although the disclosure uses coronary arteries and vessels as examples, the disclosure can be implemented to determine flow rate in other types of vessels, such as, for example, peripheral vessels, renal vessels, ocular vessels, biliary vessels, neurological vessels, or the like. In yet other non-limiting examples, processing unit 104 can execute instructions 116 to receive angiographic data (e.g., from angiographic imaging device 132, from server 138, or the like), and these data may include, but may not be limited to, angiographic images such as angiographic images 118. Other and/or additional data that may be received by processing unit 104 and may include, but may not be limited to, data received from a plurality of angiographic image frames, data received from angiographic devices and/or related devices and/or the like, metadata received from angiographic devices, and/or related devices and/or the like, and/or information received from angiographic devices, and/or related devices and/or the like.

Continuing to block 204 (“identify, by applying a computer vision algorithm with the processor, a representative image frame from the plurality of image frames”), a representative image from the plurality of images frames is identified. In some examples, processing unit 104 can execute instructions 116 to identify an image frame from the angiographic images 118 where the vessels are most clearly visible and/or where the contrast dye has most fully permeated the blood vessel tree. Said differently, processing unit 104 can execute instructions 116 to identify as the representative frame, the image frame from angiographic images 118, in which the highest volume of contrast dye is in the blood in the vessel tree and/or vessel(s) of interest in the field of view.

In some examples, the point of maximum “saturation” generally will correspond to the image frame with the highest level of contrast and/or brightness of the vessel structure as the contrast dye will cause the vessel structure to “light up” in the image frames. For example, where the angiographic images 118 are a cine loop, the representative frame may be selected as one of the later (although often not the last) frames in the cine loop as the later frames will have the contrast dye level being at or near a maximum in the blood flowing through the vessel or vessels of interest thereby causing the most vasculature structure to be visible in the representative image frame 140. Alternatively, or additionally, the processing unit 104 can execute instructions 116 to identify as representative image frame 140 the image frame from received angiographic image 118 where the contrast dye level is at a maximum in the blood flowing in the vessel or vessels of interest at the location under interrogation and/or of interest and/or within the field of view and therefore the representative image frame 140. In some embodiments, the representative image frame 140 is marked in the angiographic images 118 as part of the metadata of the images. In other examples, processing unit 104 can execute instructions 116 corresponding to a computer-vision algorithm to identify the frame from the angiographic images 118 where the contrast dye has most fully permeated the vasculature.

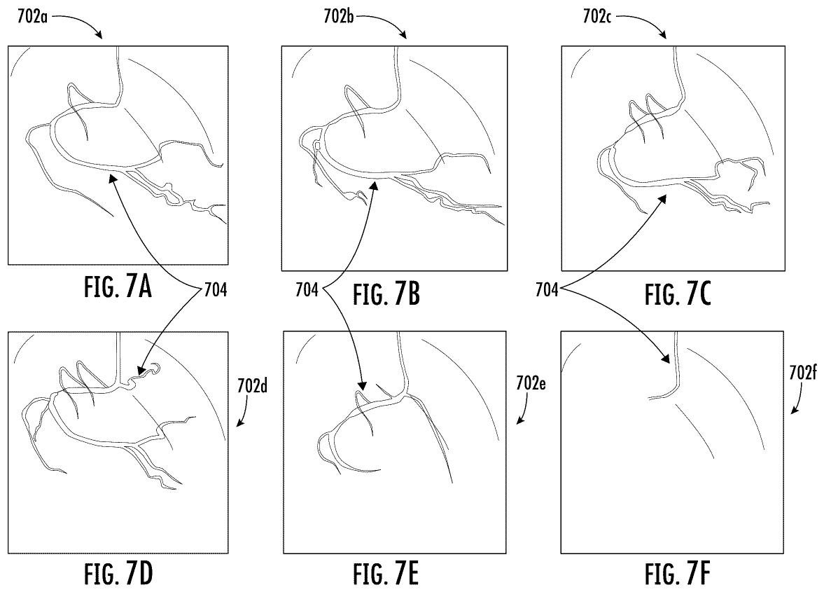

For example, turning briefly to FIG. 7A-7F, a series of angiographic images 702a through 702f are depicted. As can be seen, at least one of the frames of the images 702 corresponds to the frame of the angiographic images 702 where the highest volume of contrast dye has permeated the blood vessel tree, including but not limited to blood in the vessel 704 of the cardiac vasculature, therefore providing the clearest view of all vessels in the blood vessel tree. This frame (e.g., frame 702a, frame 702b, or the like) can be identified at block 204.

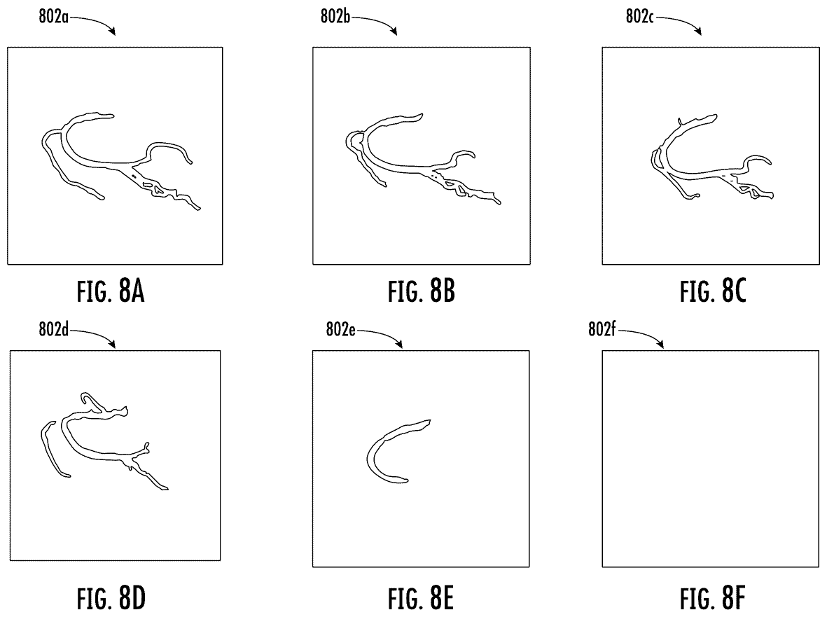

Returning to FIG. 2 and continuing to block 206 (“segment, by the processor, the plurality of image frames to isolate the vessel”), segmented 2D image frames can be generated from a 2D segmentation of the plurality of image frames received at block 202 to isolate the vessel (or vessel region) represented in the plurality of image frames. For example, processing unit 104 can execute instructions 116 to generate segmented 2D images 120 from angiographic images 118. In some embodiments, processing unit 104 can execute instructions 102 to infer segmentations for each frame of angiographic images 118 based on segmentation model 122. In other embodiments, processor 104 can execute instructions associated with an image segmentation algorithm to generate segmented 2D images 120. For example, FIG. 8A-8F depicts segmented 2D images 802a through 802f corresponding to angiographic images 702a through 702f, respectively. Specifically, FIG. 8A depicts segmented 2D image 802a that may be generated based on frame 702a depicted in FIG. 7A. Similarly, FIG. 8B depicts segmented 2D image 802b that may be generated based on frame 702b depicted in FIG. 7B; FIG. 8C depicts segmented 2D image 802c that may be generated based on frame 702c depicted in FIG. 7C; FIG. 8D depicts segmented 2D image 802d that may be generated based on frame 702d depicted in FIG. 7D; FIG. 8E depicts segmented 2D image 802e that may be generated based on frame 702e depicted in FIG. 7E; and FIG. 8F depicts segmented 2D image 802f that may be generated based on frame 702f depicted in FIG. 7F

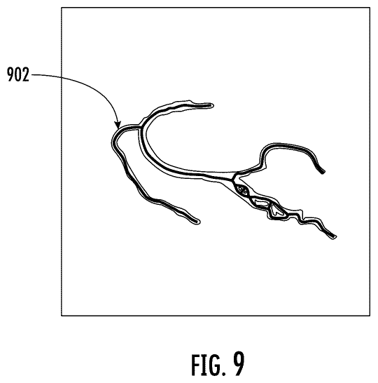

Returning to FIG. 2 and continuing to block 208 (“infer, for the representative frame, a plurality of centerline node points associated with a centerline of the vessel based in part on the segmented image frame corresponding to the representative frame”), centerline node points for the representative image frame of the plurality of image frames can be inferred from the segmented 2D image frame associated with the representative frame. For example, processing unit 104 can execute instructions 116 associated with an image processing algorithm, such as, but not limited to a computer vision skeletonization method or thinning algorithm to identify the centerline 124, which will be further described herein. Processing unit 104 can execute instructions 116 to infer centerline 124 where centerline 124 is a collection of centerline node points from the frame of segmented 2D images 120 associated with the representative image 140 and centerline identification model 126. Further, processing unit 104 can execute instructions 116 to track those centerline node points through each image frame via a tracking algorithm, such as, but not limited to a point-tracking algorithm that will be further described herein. Turning briefly to FIG. 9, centerline 902 for the vessel 704 depicted in the representative image frame 140 is shown overlaid over the frame of segmented 2D images 120 associated with the representative image frame 140.

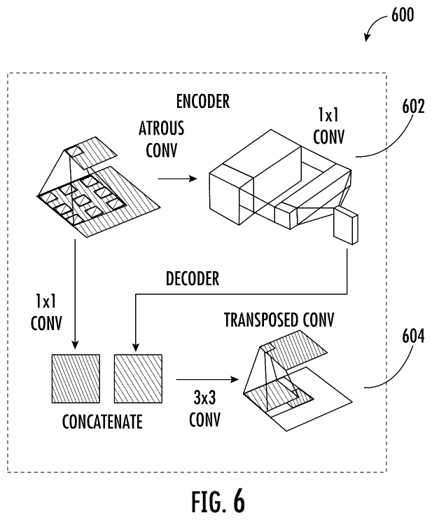

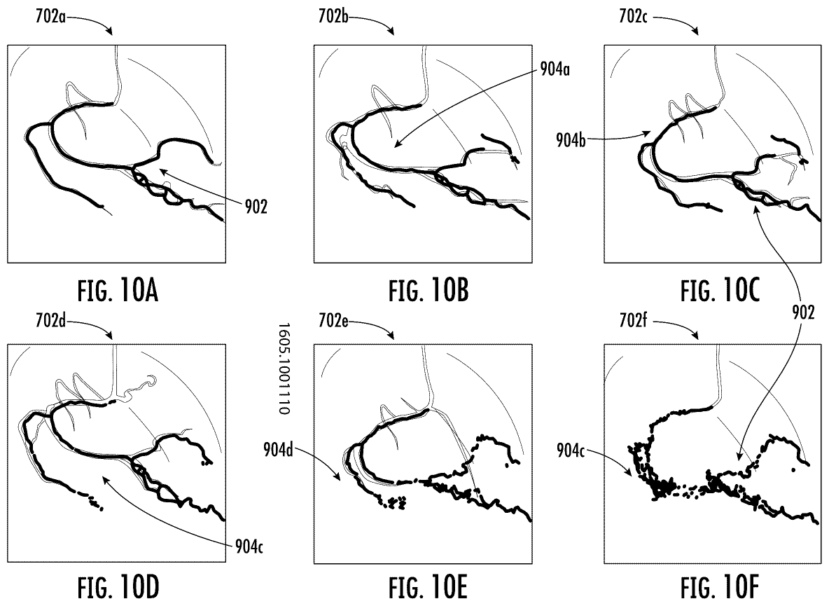

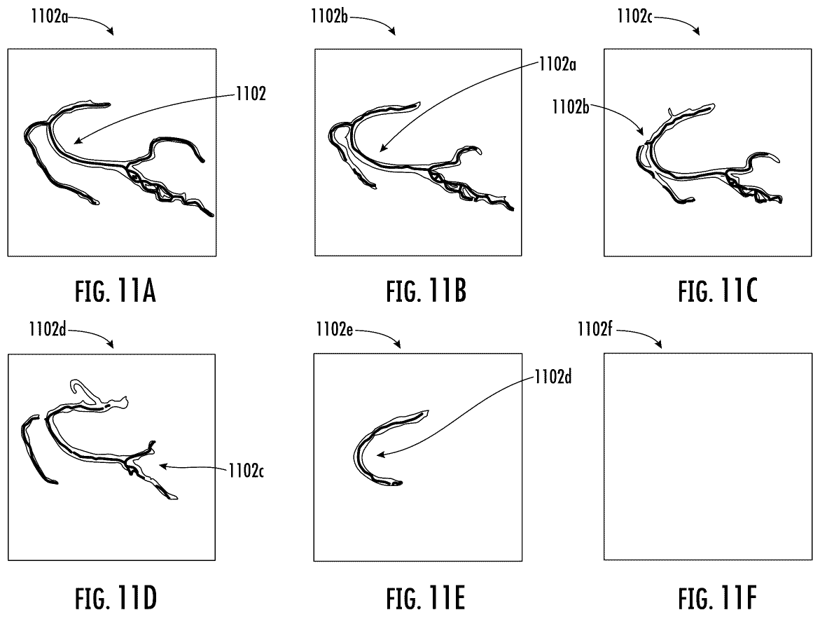

Returning to FIG. 2 and continuing to block 210 (“track, by applying a tracking algorithm with the processor, movement of the plurality of centerline node points between successive ones of the plurality of image frames”), the centerline node points can be tracked across the image frames using a point tracking algorithm. For example, processing unit 104 can execute instructions 116 to track node points of centerlines 124 from frame-to-frame across the angiographic images 118 based on a point tracking algorithm (e.g., PIP++, or the like). Point tracking algorithms of this and other examples may further include feature point tracking. For example, a feature point or feature points can be identified based on contrast changes between adjacent and/or proximate pixels to each centerline node point. For example, processing unit 106 can execute instructions 116 to determine whether a pixel proximate to a centerline node point is of slightly different value than a neighboring pixel (i.e., different color value, different contrast value, or the like), then a local feature point is generated. These local feature points may be extracted and along with other generated feature points may be mapped to the representative image frame. As will be discussed further herein, a machine learning model (e.g., see FIGS. 5 and 6) may be trained to track the identified feature points. Accordingly, processing unit 104 can execute instructions 116 to generate tracked centerlines 142 from the angiographic images 118 and the centerline 124. For example, turning briefly to FIG. 10A-10F, centerline 902 is overlaid onto angiographic images 702a through 702f and the node points of the centerline 902 are “tracked” across the images (e.g., from the representative frame 140 (e.g., angiographic image frame 702a) in order through the other images as outlined herein. In some examples, the node points of the centerline 902 can be tracked starting from the representative image frame 140 and going backwards and/or forwards in time as dictated by the location of the representative image frame 140 in the time series. This is described in greater detail below, for example, with reference to FIGS. 5 and 6.

Returning to FIG. 2 and continuing to block 212 (“align, by the processor, each frame of the plurality of segmented image frames to the segmented image frame associated with the representative image frame, based on the movement of the plurality of centerline node points between successive ones of the plurality of image frames”), each frame of the plurality of segmented image frames can be aligned to the frame of the segmented image frames associated with the representative image frame (e.g., identified at block 204) based on movement of the centerline node points across the image frames. For example, processing unit 104 can execute instructions 116 to align each frame from the segmented 2D images 120 with the frame of the segmented 2D images 120 associated with the representative image frame 140 identified from the angiographic images 118, based on movement of the node points of the centerlines 124 across the angiographic images 118 to generate stabilized segmentations 128. As described above, movement of the vessel in the stabilized segmentations 128 is removed.

Additionally, at block 212, processing unit 104 can execute instructions 116 to overlay the tracked centerlines 142 onto the stabilized segmentations 128 to generate truncated centerlines 144. For example, turning briefly to FIG. 11A-11F, centerline 902 is overlaid onto segmented 2D image 1002a while the tracked centerlines 1102a through 1102d are overlaid over segmented 2D images 1002b through 1002f, respectively. Further, the tracked centerlines 1102a, 1102b, 1102c, and 1102d are truncated based on the segmentations. For example, processing unit 104 can execute instructions 116 to truncate each tracked centerline 142 (e.g., 1102a, 1102b, etc.) where the centerline extends past the end of the vessel as represented in the segmented 2D image. Processing unit 104 can execute instructions 116 to remove node points from tracked centerlines where the nodes points extend past the end of the vessel in the segmentation based on a breadth-first-search algorithm. This is represented in FIG. 11A-11F. For example, at least some node points from tracked centerlines 1102b, 1102c, and 1102d have been truncated (or removed).

Returning to FIG. 2 and continuing to block 214 (“determine, by the processor, a flow rate of blood in the vessel based in part on a change in length of the vessel between successive ones of the image frames as represented by the truncated centerlines”), a flow rate for blood in the vessel can be determined based on the change in length of the vessel between successive ones of the image frames as represented by the truncated centerlines, which change in length is representative of the growth of saturation, or volume, of contrast dye in the vessel at each frame. For example, processing unit 104 can execute instructions 116 to (i) derive the volume of the vessel at each frame, (ii) derive a change in volume from frame-to-frame, and (iii) derive the flow rate as the change in volume over time (e.g., over the frame rate or the sampling time-period).

With some examples, processing unit 104 can execute instructions 116 to determine the blood flow based on the following example pseudo code for a “Calculate Flow Rate” function:

For each DICOM object within the vessel tree object's list of DICOM objects, a Calculate Flow Rate function may be performed to calculate increased volume within the vessel tree. Inputs into the Calculate Flow Rate function may include angiograms, angiogram data, angiographic images, angiographic metadata, point tracking results, segmentations and/or the like. The Calculate Flow Rate function may identify particles within vessel regions and/or vessel trees within each frame, accumulate centerline particles over time of analysis, and for each DICOM object, the Calculate Flow Rate function may start at the representative frame and step forward and/or backward in frames to cover all frames in the DICOM objects.

The Calculate Flow Rate function of this and other examples may connect disconnected centerline parts, calculate radius and height for each centerline point, identify new flow elements (x, y, radius), and for each new element: calculate the volume of fluid flow through the vessel tree, store volume increases for the current frame and output frame-by-frame volume increases.

The Calculate Flow Rate function of this and other examples may determine flow rate by sorting unique elements in the frame-by-frame volume increase outputs in descending order. If at least two values exist, the Calculate Flow Rate function will select the second-highest (i.e., second-largest) value as Current Increased Volume. If less than two values exist, the Calculate Flow Rate function will set Current Increased Volume to 0.

The Calculate Flow Rate function (also referred to as CalculateFlowRate function and/or Calculate Flow Rate and/or CalculateFlowRate) of this and other examples may update vessel tree inlet flow rate and convert vessel tree inlet flow rate, and/or any flow rates within any vessels within the vessel tree, to a flow rate measuring in units of m³/sec and output the final flow rate in units of m³/sec.

The following is exemplary pseudocode for computing the flow rate within a blood vessel and/or blood vessel branch and/or blood vessel tree and is listed in the table titled Algorithm 1:

Algorithm 1

```
Function CalculateFlowRate:
  For each DICOM object in the tree object's list of DICOM objects:
    1. Calculate increased volume:
      - Input: angiograms, point tracking results, segmentations
      - Process:
        a. Identify particles within vessel regions in each frame
        b. Accumulate centerline particles over time
        c. For each DICOM, we start at the representative frame and step forward in frames and
backward in frames to cover all the frames in the DICOM:
          - Connect disconnected centerline parts
          - Calculate radius and height for each centerline point
          - Identify new flow elements (x, y, radius)
          - For each new element:
            Calculate volume = π × (radius × scaling factor_{A} × pixel spacing)² × (height ×
scaling factor_{B} × pixel spacing)
          - Store volume increases for current frame
      - Output: list volume increase (frame-by-frame volume increases)
    2. Determine flow rate:
      Sort unique elements in list volume increase in descending order
      If at least two values exist:
        Select second highest value as current increased volume
      Else:
        Set current increased volume to 0
    3. Update tree inlet flow rate:
      flow rate = current increased volume / time between frames
      Convert flow rate to m³/sec
  Output: final flow rate
```

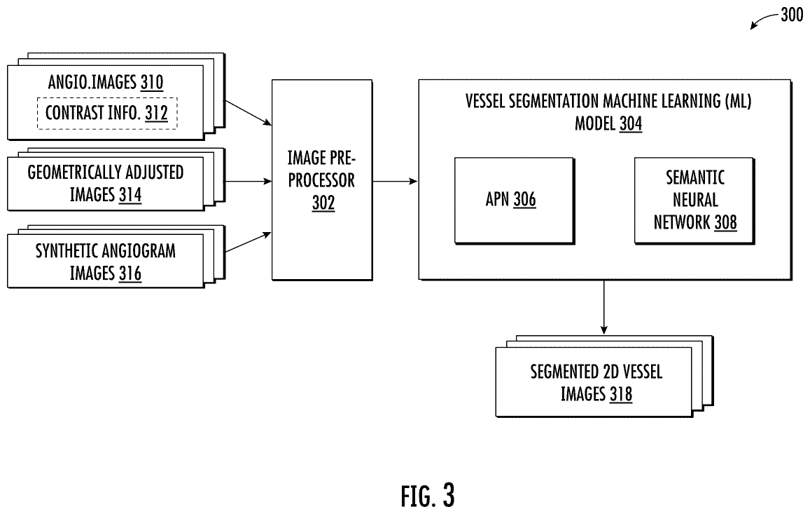

As noted above, with some embodiments, machine learning models are used as part of the overall technique. As such, an example machine learning architecture is described. FIG. 3 illustrates an example machine learning architecture 300 that may be implemented as the segmentation model 122 of computing device 102. Machine learning architecture 300 includes an image pre-processor 302 and a vessel segmentation machine learning (ML) model 304, which itself may include, for example, an angiographic processing neural network (APN) 306 and/or a semantic neural network 308.

As known in the art, machine learning (ML) is the study of computer algorithms that improve through experience. Typically, ML algorithms build a model based on sample data, referred to as training data. The model can be used to infer (e.g., make predictions or decisions without explicitly being programmed to do so).

Image pre-processor 302 can be configured to receive angiogram images 310 along with other data, such as and by non-limiting example metadata, contrast agent injection data, contrast agent data, patient data, peripheral and/or connected device data, and/or any other data and/or information applicable and known in the art. Further, in some examples, image pre-processor 302 may be configured to receive geometrically adjusted images 314. In some examples, geometrically adjusted images 314 can include any of a number of adjustments, such as flips (e.g., horizontal and/or vertical), arbitrary levels of zoom, rotation, and/or shearing. With some embodiments, the image pre-processor 302 is configured to perform various pre-processing on the received image data. For example, image pre-processor 302 can be implemented as processor executable instructions (e.g., 116) that when executed cause the processor to apply one or more image processing algorithms and/or any number of processes (e.g., denoising process, a linear filtering process, an image size normalization process, a pixel intensity normalization process, or the like) to the received images. For example, APN 306 can be configured to enhance the visibility of the contrast dye and/or stabilize the images. Further, with some embodiments, machine learning architecture 300 can be configured to generate and/or provide synthetic angiogram images 316 to image pre-processor 302. In this and other examples, machine learning architecture 300 can obtain training data sets, develop training data sets, and/or implement training data sets in association with any obtained angiogram images and/or angiogram devices and/or imaging devices and/or data to develop and/or provide synthetic angiogram images 316 to image pre-processor 302. Alternatively, or additionally, any suitable training algorithm may be used to train the machine learning architecture 300 disclosed herein.

Machine learning architecture 300 can operate in different modes, for example, a machine learning training mode and an inference mode. In machine learning training mode, the angiogram images 310, geometrically adjusted images 314, and/or synthetic angiogram images 316 may be provided to the image pre-processor 302, which is configured to pre-process the images as outlined above. In inference mode, the vessel segmentation ML model 304 (e.g., APN 306 and/or semantic neural network 308) have been trained and captured angiography image data (e.g., angiographic images 118) for a subject (e.g., patient 134) is provided to the image pre-processor 302.

In either mode described above, the output from the image pre-processor 302 is provided to the vessel segmentation ML model 304, which includes the APN 306 and/or semantic neural network 308. The vessel segmentation ML model 304 is configured to generate segmented two-dimensional (2D) vessel images from the input.

With some embodiments, the vessel segmentation ML model 304 may be a convolutional neural network (CNN). With further embodiments, the vessel segmentation ML model 304 can include two different CNNs in a staged configuration (e.g., APN 306 and/or semantic neural network 308, or the like). In some examples, the semantic neural network 308 can be based on the Deeplab v3+ architecture.

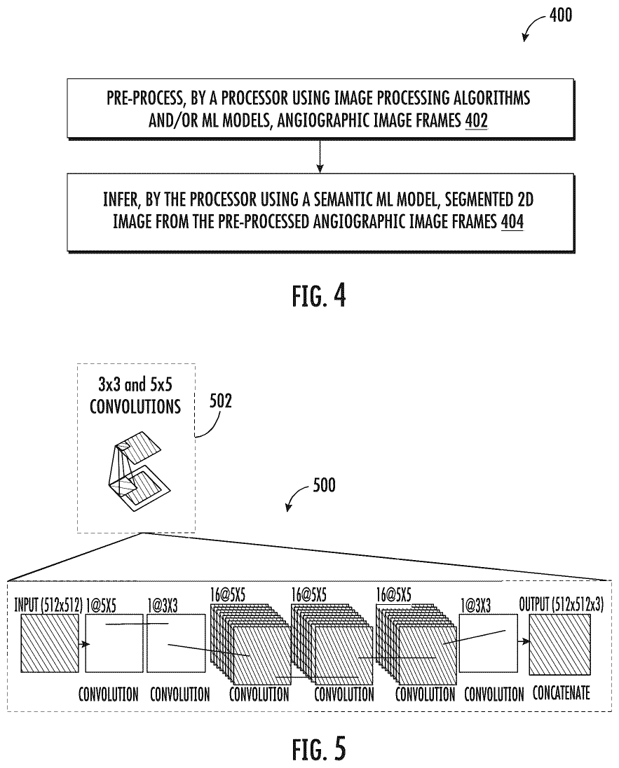

FIG. 4 illustrates a logic flow 400 that can be implemented according to the present disclosure to generate 2D segmented vessel images from angiography image data. For example, blood flow assessment system 100 can be configured to implement logic flow 400 to generate segmented 2D images 120 from angiographic images 118. As another example, logic flow 200 can perform logic flow 400 at block 206. At block 402 (“pre-process, by a processor using image processing algorithms and/or ML models, angiographic image frames”), the angiographic image frames are pre-processed through an angiographic processing neural network (APN). For example, processing unit 104 can execute instructions 116 to apply (e.g., via image pre-processor 302 or the like) image size normalization processes and/or a pixel intensity normalization process. Further, processing unit 104 can execute instructions 116 to apply (e.g., via APN 306) a non-linear filtering process, de-noising, and/or contrast enhancement. As such, objects such as catheters and bony structures can be filtered out of the angiographic images 118.

Continuing to block 404 (“infer, by the processor using a semantic ML model, segmented 2D image from the pre-processed angiographic image frames”), segmented 2D image frames are generated from the pre-processed angiographic image frames using a semantic ML model. For example, processing unit 104 can execute instructions 116 to generate segmented 2D images 120 from pre-processed versions of angiographic images 118 using semantic neural network 308.

FIG. 5 illustrates an example APN framework 500 configured with several convolutional layers 502. APN framework 500 can be configured to receive a series of angiography images (e.g., angiographic images 118, or the like) as inputs. The set of convolution layers 502 may be configured to apply linear and/or non-linear filters, which can perform contrast enhancement, boundary sharpening, and other image processing functions. FIG. 6 illustrates an example APN and DeepLabV3+ framework 600 configured with an encoder 602, which is configured to receive as input the image data output from the APN 306 (e.g., APN framework 500, or the like) and to apply atrous convolutions to the received image data.

It is to be appreciated that atrous convolution, also known as dilated convolution, is a type of convolutional operation that utilizes a parameter known as a dilation rate. The dilation rate determines how many pixels are skipped between each step of the convolution. For example, a dilation rate of 1 represents a regular convolution, while higher magnitude rates (i.e., 2, 3, 4, etc.) skip more pixels with each pass of the image frames. Unlike regular convolution, atrous convolution spaces out the filter parameters by introducing gaps between the filter parameters, controlled by the dilation rate. This process enlarges the receptive field of the filters without increasing the number of parameters, in turn allowing the connected network to capture a broader context of the input data without adding more complexity. Atrous convolution utilizes fixed kernel size with gaps controlled by dilation to preserve the input size while increasing the receptive field of the image. By increasing the receptive field of the image, atrous convolutions can enhance speed of processing by relying on fewer parameters to perform operations.

Encoder 602 can be deployed with a rate that controls the effective field of view of each convolution layer. Rates of 6, 12, and 18 may be used to affect different fields of view (i.e., areas of view) to capture different features, for example, at different resolutions. Using atrous or dilated convolutions, a dilated sample of an image, or image portion, is convolved to a smaller image. For example, the higher the rate of encoding, the greater the effective field of view (i.e., area of view) is available for each convolution layer analysis, as the area of image capture for convolution and low-level feature extraction is correlated to the rate of encoding.

Encoder 602 is configured to determine low level features using several atrous convolution strides and then to apply a 1×1 (depthwise separable) convolution to combine the outputs of the many different atrous convolutions. This produces a single matrix of features pertaining to different classes, which is input into the decoder 604 along with high level features determined from 1×1 convolutions applied to the original input image.

In some examples, the convolutions of encoder 602 may be performed using atrous or depthwise convolutions. In some examples, decoder 604 concatenates the low level and high-level features into a stacked matrix and then applies transposed convolutions to this matrix using a fractional stride size to assign class labels to each pixel based on spatial and feature information. These transposed convolutions generate a probability map which determines how likely it is that each pixel belongs to the background or vessel class.

An output layer (not shown) or a simple output function (e.g., SoftMax or the like) can be applied to the output from CNN framework 600 to generate the segmented 2D images 120.

FIG. 7A, FIG. 7B, FIG. 7C, FIG. 7D, FIG. 7E, and FIG. 7F illustrate example frames of a series of angiographic images that can be processed as outlined herein and from which a flow rate in the vessel represented in the angiographic images can be derived. For example, FIG. 7A illustrates angiogram image frame 702a showing vessel 704. Likewise, FIG. 7B illustrates angiogram image frame 702b showing vessel 704 at a different time-period. Typically, 702b is a later time period. Likewise, FIGS. 7C, 7D, 7E, and 7F illustrate angiographic image frames 702c, 702d, 702c, and 702f, respectively, captured at other times.

As can be seen from this time-series of frames, moving forward in time contrast dye can be seen in the form of increased contrast of the blood flowing through the vasculature, highlighting the structure or vascular tree of vessel 704 in increasing completeness as the image frames are captured. In other words, the contrast dye represents the blood flow as the contrast dye moves with the same direction as the blood flow as it flows through vessel 704 over the time series captured by FIG. 7A-7F. Alternatively, or additionally, the time series of moving frames may be defined and/or characterized as and/or within a time series (e.g., t=0 to n). In the example depicted in these figures, the image frame are introduced in reverse order to their order in time. For example, the frame 702a of FIG. 7A represents t=n. Although “n” is used to represent a constant in the time series any other indicator could be used (e.g., m, i, etc.). Frame 702b of FIG. 7B represents t=n−1 or the frame captured prior to the frame 702a (e.g., frame t=n). Similarly, frame 702c, 702d, 702c, and 702f of FIGS. 7C-7F may represent frames captured at times t=n−2, t=n−3, t=n−4, and t=n−5. Continuing with this example, n could equal 5 or 6 in this example. As such, frame 702f could be captured first at time t=0 (or t=1) while frame 702e is captured next, at time t=1 (or t=2), etc.

As noted above, logic flow 200 can include block 204 in which a representative frame from angiographic images 118 is identified. If angiographic images 118 included angiogram image frames 702a through 702f, processing unit 104 could execute instructions 116 to identify angiogram image frame 702a (or 702b, or the like) as the representative frame 140.

FIG. 8A, FIG. 8B, FIG. 8C, FIG. 8D, FIG. 8E and FIG. 8F illustrate example segmented 2D image frames that may be generated as outlined herein. For example, processing unit 104 can execute instructions 116 to perform a segmentation on all angiographic image frames 118 (e.g., 702a through 702f, or the like). For example, FIG. 8A illustrates segmented 2D image 802a corresponding to angiogram frame 702a from FIG. 7A. Likewise, FIG. 8B illustrates segmented 2D image 802b corresponding to angiogram frame 702b from FIG. 7B. Likewise, FIGS. 8C, 8D, 8E, and 8F illustrate segmented 2D images 802c, 802d, 802e, and 802f corresponding to angiographic image frames 702c, 702d, 702c, and 702f, respectively. These segmentations can be referred to using the same time series notation used for the corresponding image frames (e.g., t=n, t=n−1, etc.).

As outlined above, a vessel centerline 124 can be determined for the representative frame 140 and the centerline 124 tracked across all frames. For example, processing unit 104 can execute instructions 116 to determine the vessel centerline through a computer vision skeletonization method applied to the segmented 2D image frame 120 associated with the representative frame 140. For example, FIG. 9 illustrates centerline 902 that can be identified as outlined herein. In a particular embodiments, processing unit 104 can execute instructions 116 to identify frame 702a as the representative image frame, generate a skeletonization of frame 802a where frame 802a is the 2D segmentation of the representative image frame 702a, and identify centerline node points from the skeletonization of frame 802a.

An example computer vision skeletonization method and/or algorithm may skeletonize the image to extract a centerline from the image, such as a centerline of a segmented vessel from a segmented image. In digital image processing, skeletonization is a process that reduces binary objects (i.e., file or data structures representing a blood vessel or the like) to a 1-pixel-wide representation. Skeletonization works by making successive analytical passes of the image of interest (e.g., of a blood vessel or the like). On each processing pass, border pixels are identified and removed on the condition that they do not break the connectivity of the corresponding object. For instance, a computer vision skeletonization method and/or algorithm of the present disclosure may make myriad passes over a blood vessel image, removing pixels that are not representative of the vessel centerline until the vessel centerline is revealed. The centerline of the vessel may be skeletonized to, e.g., a 1-pixel width, a 2-pixel width, a 3-pixel width, a 4-pixel width, or may be reduced to a width of 5 or more pixels.

Two common algorithmic methods of skeletonization are typically practiced; these two methods may be employed using the principles of the present invention in a non-limiting capacity. One method known in the art is “Zhang's Method of Skeletonization” or the “Zhang-Suen Thinning Algorithm”. Zhang's Method operates by making successive passes of an image, and with each pass removing pixels on the desired object's borders (i.e., the object a user wishes to isolate from the image). The image is correlated with a mask that assigns each pixel a number in the range of 0-255, corresponding to each possible pattern of its 8 neighboring pixels. A look-up table is then used to assign the pixels a value of 0, 1, 2, or 3, which are selectively moved during image-pass iterations.

Another method known in the art is “Lee's Method of Skeletonization”. Lee's Method operates by using an octree data structure to examine a 3×3×3 neighborhood array of a pixel. The algorithm proceeds by iteratively sweeping over the image and removing pixels at each iteration until the image stops changing. Each iteration consists of two steps: first, a list of candidates for removal is assembled; then, pixels from this first list are rechecked sequentially to better preserve connectivity of the image.

The disclosure further provides that the centerline can be tracked from frame-to-frame. For example, the centerline 902 shown in FIG. 9 can be overlaid onto the representative frame 140 and feature points may be tracked by a point-tracking algorithm (e.g., PIPs, or the like) to track the centerline from frame-to-frame. For example, processing unit 104 can execute instructions 116 to apply a point-tracking algorithm on angiogram image data to track the centerline node points through the time series of image starting at the representative frame (e.g., frame 702a having a time of t=n, or the like) and continuing backward and/or forward in time to capture all frames in the series.

The point-tracking algorithm can be applied to assign a set of tracked points for each angiographic image frame. These tracking points can then be superimposed onto each angiographic image frame as red tracking points which are tracked from frame to frame and can be tracked through corresponding segmented image frames. In this and other non-limiting examples, the tracking points may be assigned an alternate color, or an alternate series of colors, including but not limited to blue, orange, green, purple, pink, violet, cyan, magenta, fuchsia, white, black, brown, off-white, tan, beige, yellow, red-orange, turquoise, teal, or any like color or any combination or mixture of the colors.

In an example, after red tracking points have been superimposed onto the corresponding segmented image frames, any red tracking points that reside beyond the trailing boundaries of the isolated vessels in the segmentation may be removed by using a depth first search (DFS) algorithm. The DFS algorithm removes points that are beyond where the contrast dye has progressed but avoids removing tracking points that aren't at the edge of the flow but somehow fall slightly out of the isolated vessels. In other words, the DFS algorithm (or the like) may remove tracking points that are detected beyond the boundary (i.e., outer bound of vessel) of the blood vessels that are chosen to be isolated.

FIG. 10A, FIG. 10B, FIG. 10C, FIG. 10D, FIG. 10E and FIG. 10F illustrate examples of tracked centerlines as outlined herein. For example, FIG. 10A illustrates the centerline 902 that can be identified as outlined above overlaid onto angiogram image 702a (e.g., the representative frame 140). Processing unit 104 can execute instructions 116 to track the points of the centerline 902 as outlined above. This is graphically illustrated by tracked centerlines 904a, 904b, 904c, 904d, and 904c shown in FIG. 10B, FIG. 10C, FIG. 10D, FIG. 10E, and FIG. 10F, respectively.

The processing unit 104 can further execute instructions 116 to align the 2D segmentation of each angiographic image frame to the 2D segmentation of the representative image frame to form stabilized images of the vessel and/or surrounding features. This increases the accuracy and efficacy of flow rate calculation as these stabilized images control for patient breathing, irregular heartbeat, or motor movements of the patient that may cause an angiographic image frame of a vessel or other similar structure to destabilize. The process of mapping features and data points may be an iterative process and may start with a representative frame, and then align features and data points from a frame (e.g., frame 802b at time t=n−1) to a frame (e.g., frame 802a at time t=n) based on movement of the centerline node points between these frames (e.g., between frame 802a and 802b) to align each frame of the plurality of segmented image frames to the segmented frame associated with the representative image frame. Alternatively, or additionally, in other non-limiting examples, the process of mapping features and data points may be an iterative process and may start with a representative frame, and align features and data points from a frame (i+1) to a frame (i) based on movement of the centerline node points between the frame (i+1) and the frame (i) to align each image frame of the plurality of image frames to the representative frame. In yet other non-limiting examples, the process of mapping features and data points may be an iterative process that iterates both successively and regressively. In other words, the process of mapping features and data points may be an iterative process that iterates through successive frames (i.e., i+1, i+2, i+3, etc.), previous frames (i.e., i−1, i−2, i−3, etc.), and/or both, and may perform this process simultaneously, sequentially, concurrently, or in any combination or permutation of the aforementioned.

With some examples, the point-tracking algorithm may track feature points from raw angiogram video frames. Feature points may include, but are not limited to vessel deformities, vessel occlusions, vessel concavities, vessel convexities, vessel strictures, vessel irregularities, and/or the like.

In this and other examples, the point tracking algorithm may select several tracking points based on the received angiogram images. The number of tracking points selected by the point tracking algorithm may also be influenced by the data provided from the angiogram images. In yet other non-limiting examples, multiple point tracking algorithms may be employed to track points, feature points, or other trackable features of a vessel, lumen, or other bodily structure.

The input of any of the point tracking algorithms applied herein may be a set of points in an X-Y-coordinate system which represent a list of pixels. The output of any of the point tracking algorithms applied herein may be a list of pixels presented in the correct order on an X-Y-coordinate system. When the centerline is truncated as described herein, the truncated parts of the centerline are deleted from the point tracking data. The point tracking algorithms of this and any other examples may apply more tracking points given the fidelity of data received. In other words, if higher resolution data is provided to the point tracking algorithms of the disclosure, the point tracking algorithms may yield more tracking points and/or more feature points.

As outlined above, generating stabilized images 128 can further involve truncating the tracked centerlines based on the corresponding segmented 2D image. FIG. 11A, FIG. 11B, FIG. 11C, FIG. 11D, FIG. 11E, and FIG. 11F illustrate the centerline 902 overlaid over the segmented 2D frame associated with the representative frame 140 (e.g., frame 802a). Further, these images illustrate truncated centerlines 1102a through 1102d overlaid over corresponding segmented 2D image frames (e.g., 802b through 802e, respectively). As outlined above, tracked centerlines can be truncated based on the associated segmented 2D image (or the stabilized segmented image). As such, where the tracked centerlines extend past the segmented vessel represented in the segmented 2D images, the centerlines can be truncated to form truncated centerlines by preserving the original coordinates of all points that are not removed by a depth first search (DFS) algorithm. Truncated centerlines 1102a, 1102b, 1102c, and 1102d are depicted in FIGS. 11B, 11C, 11D, and 11E respectively. It is to be appreciated that the segmentation 802f associated with image frame 702f is entirely black, indicating that the vessel structure is not visible or that no contrast dye has been absorbed or drawn into the vessel by blood flow. Accordingly, all points from the tracked centerline as truncated and the truncated centerline does not exist or is null.

As noted above, the processing unit 104 can execute instructions 116 to calculate flow rate based on the stabilized 2D segmentations. Flow rate may be derived by calculating the radius of the vessel from the centerline of the vessel by performing a pixel spacing calculation to infer the distance between the centerline of the vessel and the nearest boundary point of the vessel, which equals the radius of the vessel at that point. In some instances, the radius may be defined and/or determined as the number of pixels extending in a direction, for example, a radial direction. In other words, the number of pixels between the inferred center of a vessel to an inferred boundary of a vessel (i.e., interior vessel wall) may be quantified and/or calculated to determine and/or define the radius. Since the distance between the inferred centerline point to the nearest point in the vessel boundary is equal to the radius of the vessel, the inferred radius may be converted into a cross-sectional area due to the known mathematical relationship between the radius and cross-sectional area of a circular cross-section such as the cross-section of a blood vessel. From there, the cross-sectional area of the vessel may be multiplied by the vessel centerline length to determine the volume of blood flowing through the vessel. The volume may be divided by the image frame rate to arrive at a calculated flow rate of blood flowing through the vessel.

As detailed above, the volume of the vessel (e.g., based on a diameter of the vessel at each node point, or the like) can be derived and a change in volume between frames can be determined. As such, the flow rate can be represented as the change in volume over time. With some embodiments, processing unit 104 can execute instructions 116 to plot the flow over time.

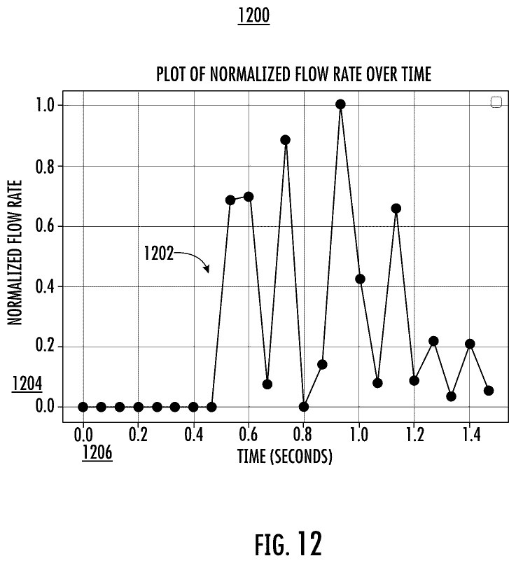

FIG. 12 illustrates a graph 1200 plotting the flow rate over time 1202 with the normalized flow rate shown on the y-axis 1204 and time (in seconds) shown on the x-axis 1206. In some embodiments, processing unit 104 can execute instructions 116 to normalize the flow rate over time 1202 (e.g., between 0 and 1, or the like). With some embodiments, the flow rate may be determined quantitatively as described herein, and then normalized. In other words, the flow rate may be determined by being derived in units of volume per unit time (i.e. m³/sec, m³/hr, cm³/sec, cm³/hr or the like) and then normalized to a value set range (e.g., between 0 and 1, or the like).

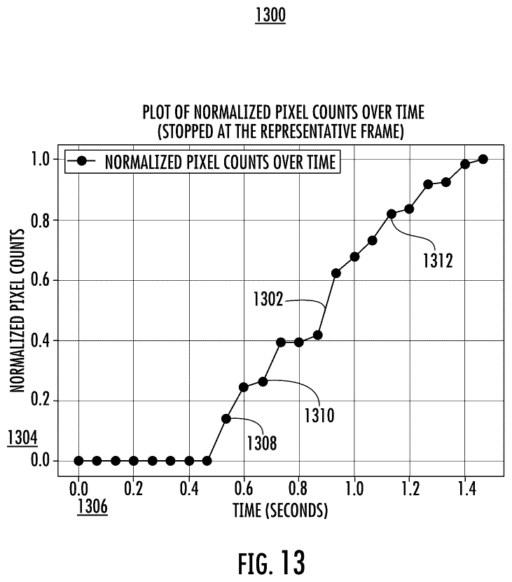

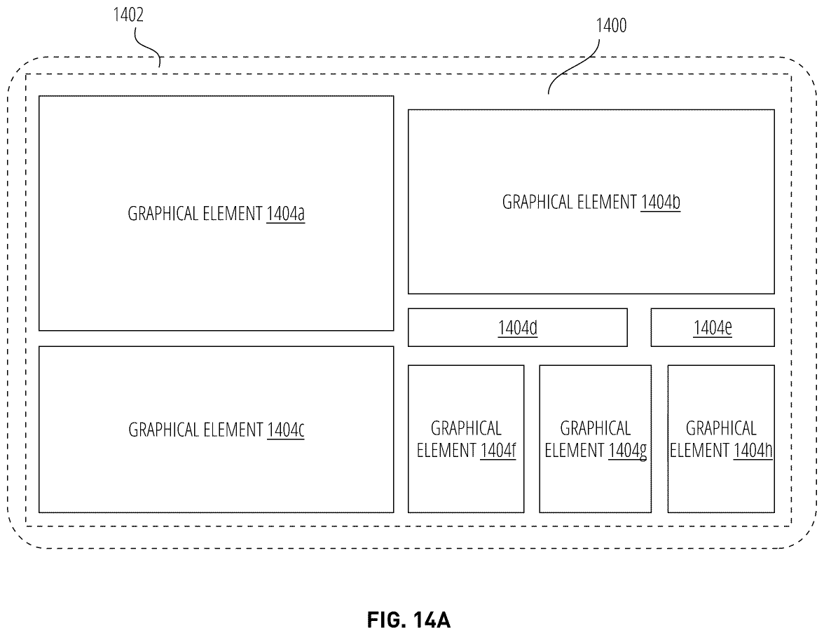

FIG. 13 illustrates a graph 1300 plotting normalized pixel counts over time 1302, with the normalized pixel counts shown on the y-axis 1304 and time (in seconds) shown on the x-axis 1306. In some embodiments, processing unit 104 can execute instructions 116 to normalize the pixel counts over time 1302 (e.g., between 0 and 1, between 0 and 10, logarithmically, and/or the like). With some embodiments, the plot of normalized pixel counts over time 1300 may display normalized pixel counts at a specified time and/or stopping point within a representative frame. As shown in FIG. 13, pixel count points 1308, 1310, 1312 (and additional pixel count points not numbered) may be captured and plotted relative to time. In this and other examples, the x-axis may denote time in seconds, however other units and/or measures of time are contemplated, including but not limited to milliseconds, microseconds, and other known units, quantities, and/or measurements.

In other examples processing unit 104 can execute instructions 116 to generate a report comprising indications of the normalized flow rate over time. The report can be provided to a user (e.g., physician, clinic, etc.) via network 136, or the like.

Any of the processors disclosed herein may further execute instructions or computer readable media or other programming to measure slope outflow of fluid (i.e. blood) as it passes through a selected vessel, selected vessel branch, selected vessel section and/or selected vessel region. The slope of the outflow of blood is the quantification of the rate at which contrast dye clears from specific microvascular and/or vascular regions. The slope of outflow provides a sensitive measure of microvascular health. Accurately and instantaneously calculating the slope of outflow allows the methods and systems of the present disclosure to detect even minor deviations from normal flow patterns, which may be indicative of early-stage microvascular and/or vascular alterations that can suggest significant potential morbidity.

Any of the processors disclosed herein and any related components may further be configured to analyze the slope of outflow as contrast dye clears a blood vessel. The slope of outflow is a quantitative measure representing the rate at which blood moves through a blood vessel. This technique provides quantitative analysis as to how blood exits the microvascular network, offering insights that are not typically visible in standard imaging modalities. The dynamic nature of this analysis allows for the observation of blood flow under varying conditions. This can highlight functional impairments that are not apparent under normal and/or homeostatic conditions.

Unlike forms of stress testing, which can be potentially deleterious to a patient and require significant patient preparation, outflow slope analysis through enhanced angiography is far less demanding on a patient and can be conducted relatively quickly. Advanced imaging technology captures detailed changes in blood flow dynamics, providing a higher resolution view of the microvascular environment than standard angiographic techniques.

Alternatively, or additionally, the techniques, systems, methods, and elements disclosed herein may be applied to data relating to a series of angiographic images.

The methods, systems and devices disclosed herein provide myriad benefits for myriad diseases, conditions and/or modalities. For diabetic patients, early detection of microvascular complications can lead to interventions that prevent serious outcomes like retinopathy, nephropathy, and neuropathy. By monitoring changes in the outflow slope, clinicians can identify early signs of endothelial dysfunction, which is a precursor to these complications.

In hypertensive patients, early microvascular changes can aid in the prediction of future cardiovascular events. Accurate and instantaneous calculation of the outflow slope can help in assessing the effectiveness of antihypertensive therapy on microvascular health, potentially guiding adjustments in treatment before more overt symptoms develop. By identifying microvascular dysfunction at an earlier stage, healthcare providers can initiate treatment interventions sooner, potentially reversing or mitigating the progression of disease.

The computer-implemented methods, systems and media of the present disclosure may be applied to treatment of the renal vasculature and/or surrounding vessels. The application of the present disclosure to renal flow analysis is also crucially beneficial, given the kidneys' significant role in filtering blood and regulating bodily fluids, electrolytes, and waste products. Kidney function largely depends on the efficiency of blood flow through the renal arteries and veins. Microvascular disease within the kidneys can lead to nephropathy and chronic kidney disease, making early and accurate diagnosis vital for effective management and treatment.

Angiogram videos focusing on the renal arteries are typically captured using standard angiographic equipment. As with angiographic techniques for other organ systems in which microvascular disease may be present, it is useful to acquire clear, high resolution images to ensure that even the smallest vascular details are visible. Using any of the frame selection algorithms of the present disclosure, frames showing optimal perfusion of contrast dye through the renal arteries and into the smaller renal vasculature are selected. Semantic segmentation is then applied specifically to isolate the renal blood vessels for detailed analysis. Once the renal vessels are segmented, a centerline for each vessel is created. This simplifies the complex renal vascular structure into manageable segments for analysis. The particle PIPs algorithm tracks the movement of the dye along these centerlines, providing real-time data on the speed and uniformity of blood flow within the renal vasculature. As the contrast progresses, the change in centerline length and the corresponding diameter of the vessels are measured to compute flow rates specifically within the renal arteries and smaller vessels. The flow rates are calculated by analyzing the change in volume over time within the segmented renal vessels. This provides quantitative data on renal blood perfusion, crucial for assessing the presence and severity of renal microvascular disease. By comparing these measurements against established norms for renal blood flow, deviations that suggest pathology can be identified and quantitatively assessed.

The innovative application of this angiographic technology to renal flow analysis is particularly impactful in diagnosing, monitoring, and managing several major renal health problems. By providing detailed and quantitative data on renal blood flow, this technique can help healthcare providers identify and treat renal conditions early and more effectively. Such renal health problems that could be addressed include but are not limited to: Renal Artery Stenosis (RAS), Chronic Kidney Disease (CKD), Acute Kidney Injury (AKI), diabetic nephropathy, hypertensive nephropathy, Renal Vein Thrombosis (RVT) and/or the like.

The computer-implemented methods, systems and media of the present disclosure may be applied to treatment of the retinal vasculature and/or surrounding vessels. Applications to retinal flow analysis also provide critical advancements in diagnosing and managing ocular conditions. The retina, being highly sensitive to blood supply variations, can manifest early signs of both local and systemic diseases. Enhanced visualization and quantitative analysis of blood flow in the retina can lead to significant improvements in the diagnosis and treatment of various retinal disorders.

In this and other examples, high-resolution angiogram videos of the retina are captured using specialized ocular imaging equipment. The focus is on ensuring that the contrast dye clearly outlines the intricate network of retinal vessels. Utilizing the proprietary frame selection algorithm, frames showing optimal contrast dye distribution across the retinal vasculature are chosen. Semantic segmentation is applied to these frames to isolate the retinal vessels for precise flow analysis. Centerlines are generated for each segmented retinal vessel, simplifying the complex vascular architecture into a form suitable for detailed analysis. The particle PIPs tracking algorithm tracks the dye movement along these centerlines, yielding real-time, quantifiable data on blood flow dynamics within the retina. The flow rates are thereby calculated by analyzing changes in vessel length and diameter over time, providing precise measurements of blood perfusion in the retina. These measurements are compared against normal flow rates to identify deviations indicative of retinal pathologies.

The application of this technology to retinal flow analysis can address several critical retinal conditions, including but not limited to diabetic retinopathy, Retinal Vein Occlusion (RVO), Age-related Macular Degeneration (AMD), Retinal Artery Occlusion (RAO), hypertensive retinopathy, and/or the like.

The technology behind enhanced angiographic analysis, particularly when focused on detailed and quantitative blood flow dynamics, has a range of potential applications across various fields in human health. As another non-limiting example, enhanced analysis of blood flow in the cerebral arteries can help in identifying occlusions or aneurysms that may lead to strokes, allowing for early intervention. The methods, systems and devices of the present disclosure may further be applied to tumor or cancerous tissue treatment. The ability to accurately measure and visualize the blood supply to tumors can inform treatment strategies, such as targeted drug delivery or surgical interventions. It also provides a means to monitor the effectiveness of anti-angiogenic drugs that aim to cut off blood supply to tumors.

For surgeries that involve significant vascular manipulation or risk, such as organ transplants or reparative surgeries, pre-surgical mapping of blood flow can help in planning the safest surgical approach. After vascular surgeries, monitoring blood flow ensures that the circulation has been successfully restored and that there are no complications like thrombosis or embolism. Enhanced angiographic analysis can further help detect reduced blood flow in the intestinal arteries, which is crucial for the diagnosis and treatment of conditions like ischemic colitis.

For other conditions that are influenced by vascular supply, such as uterine fibroids and endometriosis, understanding blood flow patterns can guide therapeutic options like embolization. For instance, in newborns and young children with congenital heart defects, precise imaging and blood flow analysis are vital for accurate diagnosis and to guide surgical interventions.

The systems, methods and devices of the present disclosure may also be utilized for pharmacometric, pharmacokinetic, pharmacodynamic or other similar analysis. By analyzing how blood flow affects drug distribution in different parts of the body, researchers can improve drug delivery systems and optimize dosages.

With some embodiments, processing unit 104 can execute instructions 116 to generate a graphical element (or graphical elements) comprising an indication (or indications) of the normalized flow rate over time for display on display 114. For example, processing unit 104 can execute instructions 116 to generate graphical elements comprising a visual representation of the blood flow rate 130 derived and/or determined according to any of the above described embodiments.

Examples of such graphical elements are given in the following figures. It is noted however, that the generated graphical element(s) may include data formatted (e.g., image frame format, or the like) such that display 114 can display the graphical elements are part of a graphical user interface. In some embodiments, the graphical element(s) can be windows (i.e., an area on the screen that displays information), container windows, browser windows, text terminals, child windows, message windows, menus, user menus, menu bars, context menus, menu extras, controls, widgets, icons, tabs, cursors, text cursors, pointer cursors, selection tools, handles, adjustment handles or the like. In this and other examples, the term and/or phrase ‘graphical element’ may be equivalent and/or synonymous with module, interface element, interface feature, module feature, module element, modular component, modular element, modular feature or any like term and/or similar term in the art.

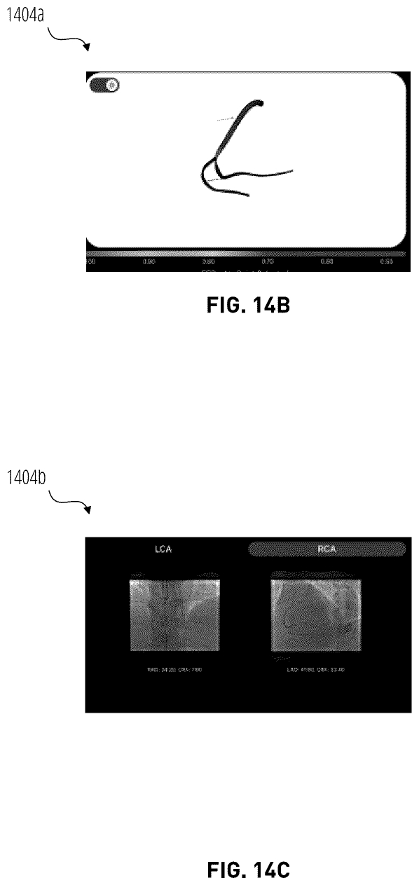

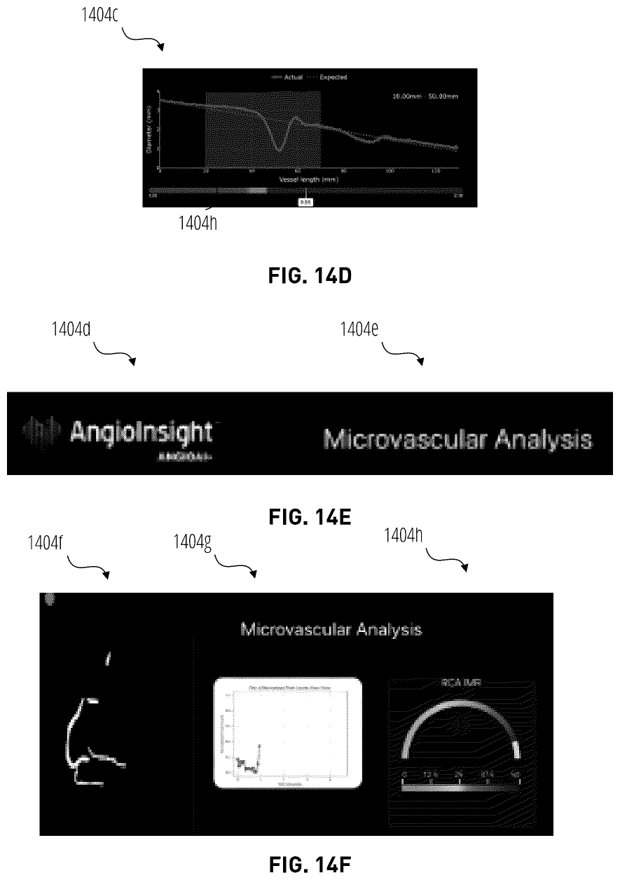

FIG. 14A displays an example graphical user interface (GUI) 1400 presented on a display 1402. The GUI 1400 can include several graphical elements 1404. Although GUI 1400 is depicted including graphical elements 1404a through 1404h; it is noted that the GUI 1400 could comprise any number of graphical elements 1404, such as, 1, 2, 3, 4, etc. In general, GUI 1400 could comprise any combination of graphical elements 1404a, 1404b, 1404c, 1404d, 1404c, 1404f, 1404g, and/or 1404h. As a specific example, GUI 1400 could comprise graphical elements 1404c and 1404h while the other depicted graphical elements form no part of the GUI !400.

In some embodiments, the positioning, orientation, size of and/or other display attributes of the various graphical elements 1404 may be manipulated, reorganized, rearranged and/or re-arrayed, for example, by a user of the device with which the GUI 1400 is displayed (e.g., microvascular blood flow assessment system 100, or the like).

The graphical elements 1404a through 1404h can be any of a variety of graphical elements or modules, which in some cases may be interactive (e.g., dynamically changed during use, able to receive input, or the like). For example, graphical elements 1404a through 1404h can include indications of blood pressure, pressure gradients, pressure flow curves, 2D and/or 3D vessel depictions or reconstructions, vessel tree depictions, angiographic imaging information, or the like.

Turning to FIG. 14B, graphical element 1404a, in the form of a vessel reconstruction module is depicted. As shown, the graphical element 1404a is configured to display a 3D vessel reconstructions, in which a patient and/or subject blood vessel and/or blood vessel tree is accurately modeled in real-time and displayed in any 3D format known in the art. In other non-limiting examples, the graphical element 1404a may be adapted and/or otherwise configured to display 2D vessel reconstructions, in which a patient and/or subject blood vessel and/or blood vessel tree is accurately modeled in real-time and displayed in any 2D format known in the art.

FIG. 14C depicts graphical element 1404b, in the form of an angiographic image playback module. Graphical element 1404b can include a button configured to toggle between displaying right carotid artery (RCA) information and left carotid artery (LCA). Further, graphical element 1404b can include visual indications of media and be adapted to playback (e.g., replay, rewind, fast-forward, pause, stop, and/or start) the media (e.g., angiographic image information, or the like). For example, graphical element 1404b can comprise visual indications of data and/or information relating to a diagnostic procedure, including but not limited to an angiographic procedure.

FIG. 14D depicts graphical element 1404c, in the form of blood flow information display. For example, graphical element 1404c can be configured to visually display indications of blood pressure, microvascular pressure, blood pressure gradient, microvascular blood pressure gradient, FFR analysis results, FFR analysis computations, and/or the like. With some examples, graphical elements 1404c and 1404b can be coupled such that the blood flow visualizations in graphical element 1404c display information for either the RCE or LCA as selected in graphical element 1404b.

FIG. 14E depicts graphical elements 1404d and 1404c, in the form of logos. For example, GUI 1400 can include graphical elements depicting a logo of the source of the GUI.

FIG. 14F depicts graphical elements 1404f, 1404g, and 1404h, in the form of widgets configured to display visual indications of the blood flow rate 130. For example, graphical elements 1404f, 1404g, and/or 1404h can be configured to display real-time parameters and/or values concerning a patient or subjects blood flow, microvasculature blood flow. For example, graphical elements 1404f, 1404g, and/or 1404h can be graphs, plots, models, gauges, or the like configured to visually display some of the information derived as outlined above.

For example, graphical elements 1404f, 1404g, and/or 1404h can be configured to display visual indications of a “diagnostic analysis” (e.g., any analysis and/or procedure that aims to achieve a prognosis, diagnosis, or determine the health status of a patient and/or subject) such as the analysis described herein. In non-limiting examples, diagnostic analysis as described herein may include but is not limited to angiographic procedures, catheterization procedures, mathematically modeled procedures, physics-modeled procedures, machine-learning (ML)-modeled procedures, AI-modeled procedures.

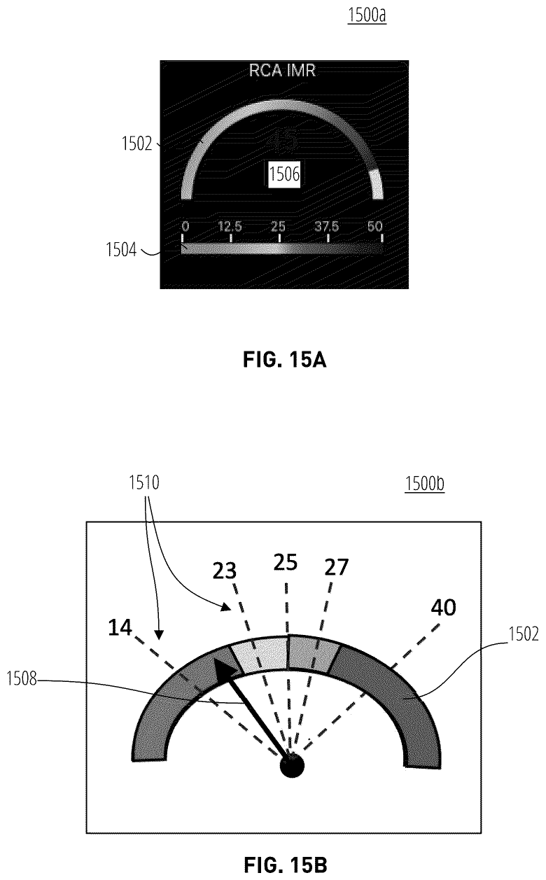

FIG. 15A shows a graphical element 1500a, which may be provided a graphical element 1404 of GUI 1400. The graphical element 1500a is in the form of a microvascular analysis gauge and is configured to display an index characterizing microvascular resistance (e.g., RCA/LAD IMR) within a blood vessel and/or related vessel structure. The graphical element 1500a comprises a gradient arc 1502, a gradient bar 1504, and an index 1506. The gradient arc 1502 and gradient bar 1504 may conform to any shape or geometry known in the art, including but not limited to a line, a dotted line, any truncated shape, and/or the like.

FIG. 15B shows a graphical element 1500b, which may be provided a graphical element 1404 of GUI 1400. The graphical element 1500b is in the form of another microvascular analysis gauge. For example, graphical element 1500b includes a gradient arc 1502 as well as needle 1508 and guidelines 1510. In general, the needle 1508 can be any visual indicator (e.g., arrow, indicia, marker, etc.) to point to or indicate real-time parameters and or values, including all real-time parameters and/or values disclosed and described herein. Guidelines 1510 may depict various levels or severity of the assessment indicated by graphical element 1500b.

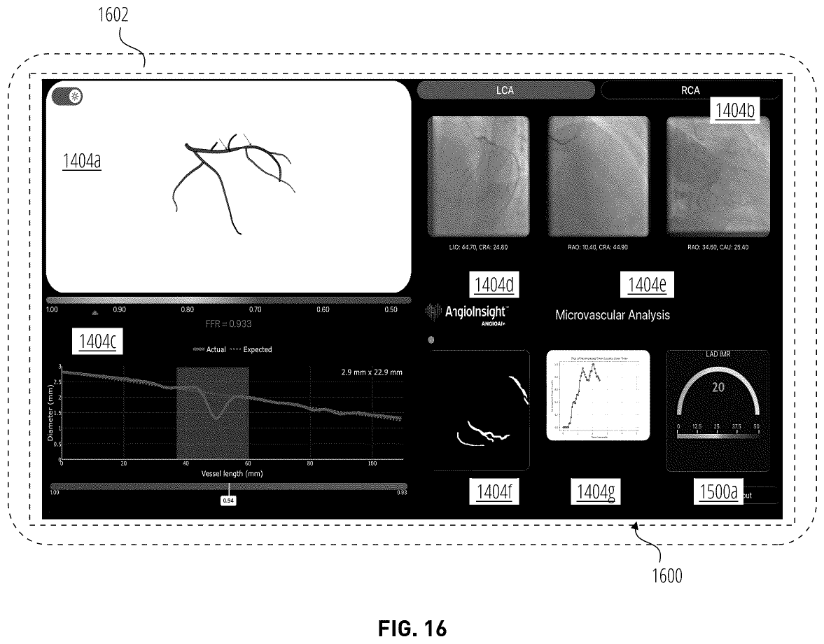

FIG. 16 displays a GUI 1600 in accordance with at least one example. In this and other examples, GUI 1600 may be displayed or presented upon a display 1602 (e.g., display 114, or the like). As shown, the GUI 1600 comprises graphical elements 1404a, 1404b, 1404c, 1404d, 1404e, 1404f, 1404g, and 1500a.

This disclosure is, in many respects, only illustrative. Changes may be made in details and arrangement of steps without exceeding the scope of the disclosure. This may include, to the extent that it is appropriate, the use of any of the features of one example embodiment being used in other embodiments.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed is:

1. A computing system configured to display visual indications of vascular blood flow, comprising:
a processor;
a display; and
memory comprising instructions that when executed by the processor cause the processor to:
generate one or more graphical elements comprising a visual indication of blood flow;
generate a graphical user interface (GUI) based in part on the one or more graphical elements; and
cause the GUI to be displayed on the display.
